(19) United States
(12) Patent Application Publication
(10) Pub. No.: US 2022/0157421 A1
(43) Pub. Date: May 19, 2022

(54) SYSTEM AND METHOD FOR FORMING AUDITABLE ELECTRONIC HEALTH RECORD

(71) Applicant: REAL-TIME ENGINEERING AND SIMULATION INC., Kanata (CA)

(72) Inventors: Bobby Chawla, Kanata (CA); Raviraj Manubhai Mangukiya, Kanata (CA); Adam Freed, Ottawa (CA); Jose Francisco Zambrano Guerrero, Ottawa (CA); Karen Angela Spreng, Ottawa (CA)

(21) Appl. No.: 17/511,719

(22) Filed: Oct. 27, 2021

Related U.S. Application Data
(60) Provisional application No. 63/113,404, filed on Nov. 13, 2020.

(51) Int. Cl. G16H 10/60; G06K 9/62; H04L 29/08

(52) U.S. Cl. CPC G16H 10/60; H04L 67/12; G06K 9/6265; G06K 9/6256

(57) ABSTRACT

A method for populating an electronic health record, and a system configured to perform this method, comprises a step of analyzing captured raw data to classify the same according to type of health information to which it pertains; and a step of converting the raw data to a format based on input type of a data field which is associated with the classified type of health information. The method features steps of determining and displaying confidence values representative of predicted accuracies of the converted data relative to the raw data and of selection of the data field receiving the converted data; and requesting user-verification that the converted data is representative of the raw data.

This application claims the benefit under 35 U.S.C. 119(e) of U.S. Provisional Application Ser. No. 63/113,404 filed Nov. 13, 2020.

### FIELD OF THE INVENTION

The present invention relates generally to a method and a system for populating an electronic health record, and more particularly to such a method and system in which the electronic health record is automatically populated with data captured by at least one data acquisition device, which data is subsequently made available to a user for review before finalizing the record.

### BACKGROUND

Conventionally, health data is manually input by a user into an electronic health record. However this is a time-inefficient process which relies on memory and recall of events after the fact, and often results in human-related transcription errors. There exists an opportunity for automated input of data into an electronic health record using electronic computing devices. However such devices are also prone to error when converting collected data for entry into the health record.

### SUMMARY OF THE INVENTION

According to an aspect of the invention there is provided a method for populating an electronic health record using a system comprising a computing device and a plurality of data acquisition devices communicatively coupled to the computing device,

wherein the computing device comprises a processor, a memory operatively coupled thereto and configured to store executable instructions thereon, and a visual display configured for displaying information to a user,

wherein the computing device is configured to receive input from the user,

wherein the electronic health record is stored on the memory of the computing device,

wherein the electronic health record comprises a plurality of data fields configured for receiving input, each data field being associated with a different type of health information about a patient,

the method comprising:

after capturing, using at least one of the data acquisition devices, raw data about the patient, classifying, using the system, for the raw data captured by a corresponding one of the data acquisition devices, said raw data according to a type of health information by analysis of said raw data;

using the system, converting the raw data captured by the corresponding data acquisition device to a form for input to a corresponding one of the data fields associated with the classified type of health information of said raw data;

using the system, displaying to the user a confidence value representative of a predicted accuracy of the converted data input to the corresponding data field relative to the raw data captured by the corresponding data acquisition device, wherein the confidence value is based on the data acquisition device with which the raw data was captured;

using the system, providing the raw data to the user for comparison to verify the converted data input to the data fields of the electronic health record; and

using the system, requesting input, from the user, to confirm that the converted data input to the data fields of the electronic health record are representative of the raw data captured by the data acquisition devices and actual types of health information associated therewith.

According to another aspect of the invention there is provided a system for populating an electronic health record, wherein the electronic health record comprises a plurality of data fields for receiving input, each field being associated with a different type of health information about a patient,

the system comprising:

a portable computing device having a processor, a memory operatively coupled thereto and configured to store executable instructions thereon, and a visual display configured for displaying information to a user;

wherein the portable computing device is configured to receive input from the user,

wherein the memory of the portable computing device is configured to store the electronic health record thereon;

a plurality of data acquisition devices communicatively coupled to the portable computing device;

wherein at least one of the data acquisition devices is arranged to be located on a body of the user so as to be worn;

wherein each data acquisition device has a processor and a memory operatively coupled thereto and configured to store executable instructions thereon to:
classify raw data captured by the data acquisition device according to a type of health information by analysis of said raw data;
convert the raw data that has been captured to a form for input to a corresponding one of the data fields of the electronic health record associated with the classified type of health information of said raw data; and
determine a confidence value representative of a predicted accuracy of the converted data to the raw data.

According to yet another aspect of the invention there is provided a method for forming a database of electronic health records containing information which is searchable, using a system comprising a computing device and a plurality of data acquisition devices communicatively coupled to the computing device,

wherein each electronic health record comprises a plurality of data fields configured for receiving input, each data field being associated with a different type of health information about a patient,

wherein each data acquisition device is configured to capture non-textual raw data,

the method comprising:

after capturing, using at least one of the data acquisition devices, raw data about a patient for a corresponding one of the electronic health records, classifying, using the system, for the raw data captured by a corresponding one of the data acquisition devices, said raw data according to a type of health information by analysis of said raw data;

using the system, converting the raw data captured by the corresponding data acquisition device to a form for input to a corresponding one of the data fields associated with the classified type of health information of said raw data;

wherein the form for input to the corresponding data field comprises one of a textual transcription of the raw data, system-selection of a predefined selectable option in a predetermined list, and system-selection of an optionally selectable field;

using the system, providing the raw data to the user for comparison to verify the converted data input to the data fields of the corresponding electronic health record; and

using the system, requesting input, from the user, to confirm that the converted data input to the data fields of the corresponding electronic health record is representative of the raw data captured by the data acquisition devices and actual types of health information associated therewith.

Preferably, there is provided a step of displaying, using the system, for the raw data captured by the corresponding data acquisition device, a confidence value to the user that is representative of a predicted accuracy of the classified type of health information of said raw data to the actual type of health information with which said raw data is associated, wherein the confidence value is based on the data acquisition device with which the raw data was captured.

There may be a step of capturing, using the system, the raw data for subsequent conversion and input to the electronic health record.

For example, capturing the raw data comprises recording speech of the user.

For example, capturing the raw data comprises recording an electromagnetically transmitted audio communication between the user and a remote entity.

For example, capturing the raw data comprises recording audio and transcribing the audio to text in real-time.

For example, capturing the raw data comprises scanning a unique identifier of a personal identification document of the patient.

For example, capturing the raw data comprises capturing an image of a label on a personal medication container of the patient.

For example, capturing the raw data comprises recording information of a medication administered by or a procedure performed by a healthcare team. In other words, capturing the raw data may comprise capturing an image of medication prior to administration to the patient.

There may also be a step of capturing, using the system, location data from at least one of the data acquisition devices.

In one embodiment, the step of displaying to the user a confidence value representative of a predicted accuracy of the converted data comprises flagging for the user's review, using a visual marker, the corresponding data field into which the converted data was input when the confidence value is below a prescribed threshold value of the system.

The prescribed threshold value may be 100%. That is, the prescribed threshold value is system-defined as 100%.

Alternatively, the prescribed threshold value is defined by input to the system.

Preferably, there is provided a step of checking, using the system, if one of the data fields of the electronic health records contains converted data from multiple ones of the data acquisition devices, and
if one of the data fields contains converted data from multiple ones of the data acquisition devices:
   comparing the converted data from the multiple data acquisition devices to determine whether information represented by said converted data is consistent, and
   if the corresponding data field is determined to contain information that is inconsistent, flagging for the user's review, using a visual marker, the corresponding data field.

When the raw data captured by the corresponding data acquisition device is audio data, at least one of steps of (i) classifying the raw data according to a type of health information associated therewith and (ii) converting the raw data to a form for input to a corresponding one of the data fields associated with the classified type of health information may comprise analyzing, using the system, sentences detected in the raw data to determine at least one of a context and a speaker of a situation represented by the raw data.

Analyzing sentences detected in the raw data to determine a speaker may comprise analyzing voice patterns in the detected sentences to distinguish speakers.

Analyzing sentences detected in the raw data may comprise classifying sentence types of the detected sentences.

Analyzing sentences detected in the raw data may comprise categorizing each sentence according to a predetermined list of the types of health information about the patient.

Analyzing sentences detected in the raw data may further comprise grouping sentences detected as questions and sentences detected as related responses and correlating the sentences in the grouping to determine the context of the situation represented by the raw data.

When the raw data captured by the corresponding data acquisition device is audio data and when the form of the converted data is text, there may be a step of checking, using the system, at least one of spelling and grammar of said converted data for classifying context of the situation represented by said raw data.

When the raw data captured by the corresponding data acquisition device is audio data, the step of providing the raw data to the user for comparison to verify the converted text may comprise providing an audio clip associated with the said data for playback by the user.

When the raw data captured by the corresponding data acquisition device is image data, the step of providing the raw data to the user for comparison to verify the converted text may comprise displaying to the user an image associated with said raw data.

There may be a step of automatically attaching to the electronic health record data collected from a medical diagnostic device that is operatively communicated with the system.

There may be a step of deleting, using the system, the raw data after user-confirmation of the validity of the data fields of the electronic health record.

Requesting input, from the user, to confirm that the converted data input to the data fields of the electronic health record are representative of the raw data captured by the data acquisition devices and actual types of health information associated therewith may comprise receiving input, from the user, to correct the converted data to be representative of the raw data.

There may be a step of training the system based on corrections to the converted data made by the user.

In one embodiment, the confidence value representative of a predicted accuracy of the converted data input to the corresponding data field relative to the raw data captured by the corresponding data acquisition device is based on an initial predetermined value associated with the corresponding data acquisition device and previous user confirmations of converted data based on raw data captured by the corresponding data acquisition device.

In one embodiment, converting the raw data captured by the corresponding data acquisition device to a form compatible for input to a corresponding one of the data fields is performed by the corresponding data acquisition device.

In one embodiment, the confidence value representative of a predicted accuracy of the converted data input to the corresponding data field relative to the raw data captured by the corresponding data acquisition device is provided to the computing device by the corresponding data acquisition device.

### BRIEF DESCRIPTION OF THE DRAWINGS

The invention will now be described in conjunction with the accompanying drawings in which:

FIG. 1 is a schematic diagram of a system according to the present invention;

FIG. 2 is a schematic diagram of an electronic health record;

FIG. 3 is a flowchart of method steps according to the present invention;

and

FIG. 4 shows a scenario in which raw data may be concurrently acquired from multiple sources.

In the drawings like characters of reference indicate corresponding parts in the different figures.

### DETAILED DESCRIPTION

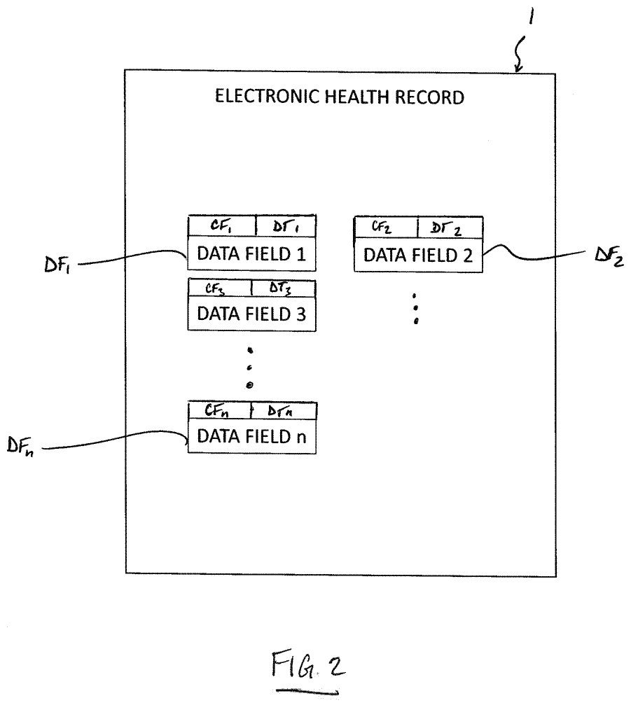

The accompanying figures show a system and method for populating an electronic health record 1 (FIG. 2).

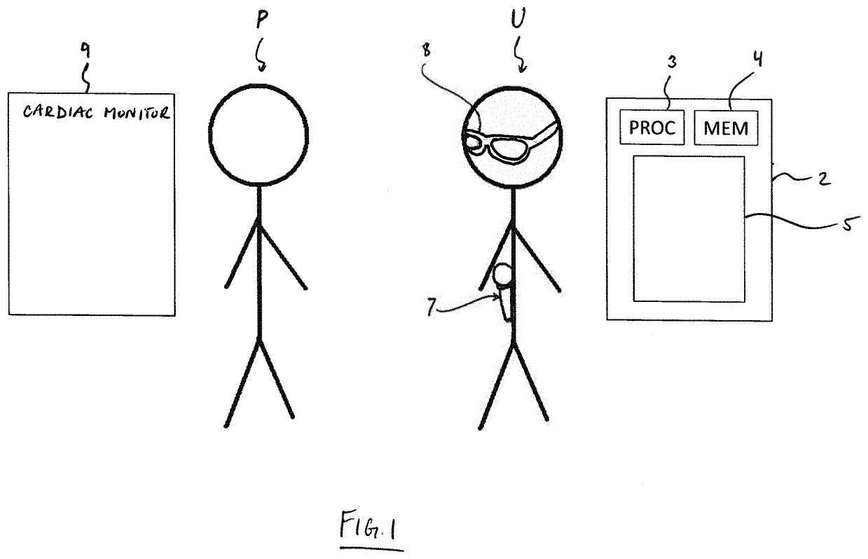

Referring to FIG. 1, the system thereof generally comprises the following components:

a conventional computing device 2 such as a tablet computer comprising a processor 3; a memory 4, that is a non-transitory readable storage medium, which is operatively coupled to the processor 3 and configured to store executable instructions thereon; and a visual display 5 configured for displaying information to a user U and configured, in the illustrated embodiment, to receive input from the user U such that the computing device 2 is generally configured to receive input from the user U; and

a plurality of data acquisition devices, such as those indicated at 7 through 9, which are configured to collect data of a prescribed format about a patient P and which are communicatively coupled to the computing device 2 for transmitting the collected data thereto.

It will be appreciated that the electronic health record 1 is at least temporarily stored on the memory 4 of the computing device 1 as the health record is populated by the system, but thereafter the health record may be stored on a remote server (not shown) to which the computing device 2 is communicatively coupled, for example, wirelessly over a terrestrial data communication network.

Generally speaking, the data acquisition devices such as 7,8 and 9 of the system are configured to collect raw data about the patient P in the format of at least one of audio data, image data and location. For example, raw audio data that can be collected may include speech of the user, and an electromagnetically transmitted audio communication between the user and a remote entity such as a dispatch center. Audio data may be transcribed to text in real-time (that is, transcribed substantially simultaneously as the data is collected) and stored in text format. In another example, raw image data that can be collected may include a scanned unique identifier of a personal identification document of the patient, a photographed label on a personal medication container, and a photograph of medication prior to administration to the patient. In a further example, raw location data that can be collected may include global positioning coordinates.

Thus the data acquisition devices generally include at least one audio capture device, such as a microphone indicated at 7, and at least one image capture device, such as smart glasses or a camera indicated at 8. Preferably there is also at least one location capture device, which may be one of the data acquisition devices which is configured to collect at least one of audio and image data so that the same device is also further configured to collect location data.

Typically, the data acquisition devices each have a processor and a memory operatively coupled thereto and configured to store executable instructions thereon, such that each acquisition device locally performs the steps of data capture or acquisition and processing before transmitting data to the computing device 2 for entry or input into the health record 1.

When the user U is deployed in a mobile data acquisition scenario, the computing device 2 is portable and one or more of the data acquisition devices are arranged to be located on a body of the user so as to be worn. This enables data acquisition in real-time as the raw data is initially made available to the user, in other words the aforementioned system configuration including wearable data acquisition devices facilitates data collection ‘on the go’.

In some configurations of the system, the system data acquisition devices may include a medical diagnostic device such as a defibrillator or a cardiac monitor 9 that is operatively communicated with the system, in particular the health record-storing computing device 2.

Referring to FIG. 2, the electronic health record 1 comprises a plurality of data fields, such as DF₁ and DF₂ through DFₙ, each configured for receiving input.

Each data field is associated with a different type of health information about the patient P. Examples of types of health information about the patient include patient name, patient birthdate, existing medical conditions, and symptoms being experienced. The input to a respective one of the data fields comprises one of a free-form textual input, selection of one or more predefined selectable option in a predetermined list, and an optionally selectable box.

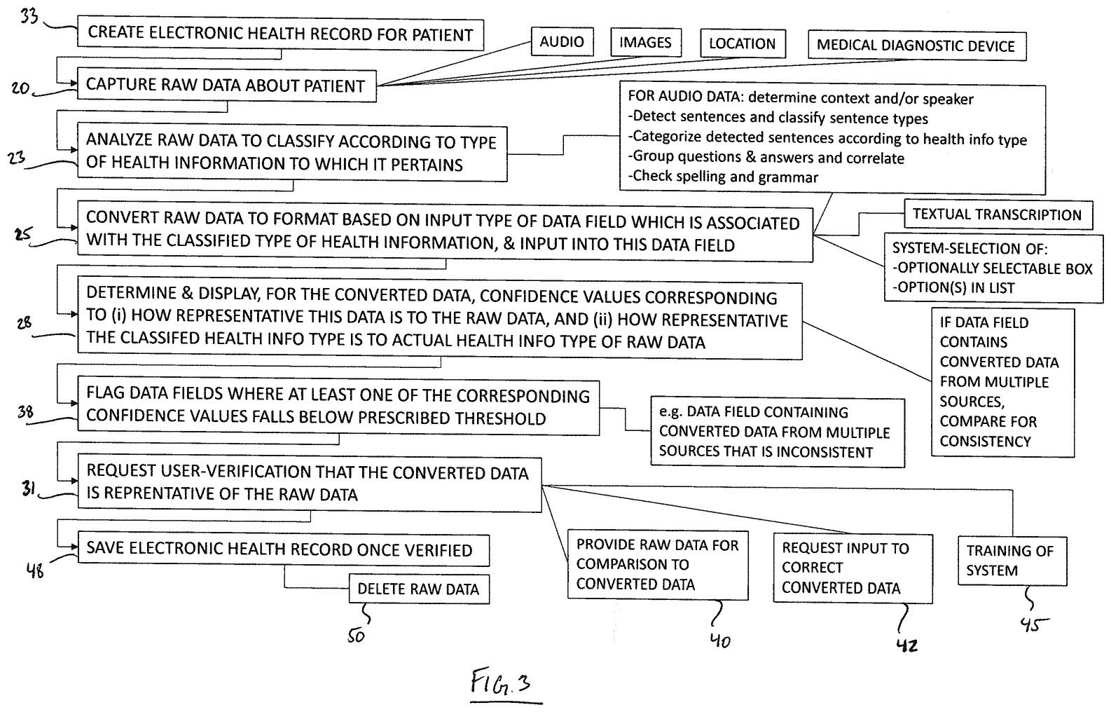

The system is configured to perform the following steps, as shown in FIG. 3:

capture raw data using at least one of the data acquisition devices such as any one of those indicated at 7-9 for subsequent conversion and input to the electronic health record 1, as at step 20;

classify the raw data captured by a corresponding data acquisition device according to a type of health information by analysis of the raw data, as indicated at step 23;

convert that raw data to a form for input to a corresponding one of the data fields of the electronic health record associated with the classified type of health information of said raw data, as indicated at step 25; and

determine and display a confidence value representative of a predicted accuracy of the converted data to the raw data, as indicated at step 28; and

request user-verification that the converted data is representative of the raw data, as indicated at step 31.

It will be appreciated that steps 23, 25 and 28 are performed for each raw data source, that is for the raw data captured by each data acquisition device, such that when the system comprises more than one data acquisition device the aforementioned steps are carried out more than once. On the other hand, the user-verification step 31 does not have to be conducted serially after conversion and input of the raw data from each data acquisition device but may be left as a single final step once all raw data has been processed and input or entered.

Generally speaking, an initial step preceding capturing of the raw data at 20 is to create the electronic health record 1 for the patient P, which is to be subsequently populated with information which is to be collected, as at step 33. In at least one embodiment the electronic health record 1 is stored on the computing device 2 during the step of populating the same. This information to be input or entered into the health record is derived from raw data of various formats which is captured by data acquisition devices such as those indicated at 7-9.

The step of capturing raw data about the patient P at 20, which data can be of various formats, may therefore include any one of the following:

recording speech of the user U, for example by microphone 7;

recording an electromagnetically transmitted audio communication between the user and a remote entity, for example a telephone call between a paramedic and a medical doctor or a radio communication between the paramedic and a dispatch center, which may be performed by microphone 7 or by another audio recording device coupled to a communication network facilitating the aforementioned audio communication;

scanning a unique identifier of a personal identification document of the patient P, for example scanning a barcode of a health card, which may be performed by smart glasses 8;

capturing an image of a label on a personal medication container of the patient P, for example a label from a pharmacy which supplied prescription drugs to the patient;

capturing an image of medication to be administered to the patient, for example a syringe filled with liquid medicine (prior to administration thereof) or pills/tablets of oral medication before they are given to the patient, which images can be used to determine (using the system) amount of medication administered to the patient for recordation in the electronic health record;

capturing an image of a medical document;

capturing location data; and

automatically attaching to the electronic health record data collected from a medical diagnostic device such as cardiac monitor 9 that is operatively communicated with the system.

It will be appreciated that raw data of audio format may also be transcribed to text in real-time such that both the audio and textual transcription, the latter of which is easier to audit, are made available to the user U at the verification step 31.

The step of classifying the raw data derived from a single data acquisition device by type of health information to which it pertains, as at 25, may involve different analysis techniques based on the format of the raw data.

For example, for raw audio data, the analysis comprises detecting sentences and determining based on the detected sentences at least one of a context and a speaker of a situation that is represented by the raw data. Also, the detected sentences are classified by sentence type, for example paramedic questions paired with patient responses and ignoring patient questions paired with paramedic responses that are irrelevant. Based on the detected sentences, sentences detected as questions are grouped with sentences detected as related responses, usually based on pairing a question with a statement which follows or trails the question, to determine context of the situation represented by the raw data. Furthermore, each detected sentence is categorized according to a predetermined list of the types of health information about the patient, which helps to determine the context of the situation and ultimately the data field of the electronic health record where the raw data is to be input.

To classify and/or confirm context, when the audio data has been converted to text, the system checks at least one of spelling and grammar of the text.

In another example, for raw image data, the analysis comprises applying optical character recognition to determine the type of health information with which the raw data should be classified. Additionally or alternatively, this analysis comprises accessing or mining a database linked to a unique identifier that is scanned.

To classify and/or confirm speakers, the system analyzes voice patterns in the detected sentences to distinguish a plurality of speakers. This is achieved, for example, by analyzing frequencies of speech in the detected sentences and classifying a similar set or range of frequencies as belonging to a single speaker. Once the system is able to discriminate between the speakers, it is enabled to decide whether to classify raw data associated with a speaker who is determined to be a bystander of the situation.

Once the raw data has been classified according to health information type, the system can proceed to convert the raw data to an appropriate form for input to one or more of the data fields in the electronic health record which are associated with the classified type of health information. Thus, forms or formats of input for the data fields include free-form text, which is suited for receiving textual transcription of raw data; a predetermined list of predefined selectable options; and an optionally selectable box or field. In the case of a data field which receives free-form text, conversion comprises transcribing the raw data to text (if this already has not been performed at an earlier step or stage). In the case of a predetermined list of predefined selectable options, conversion comprises system-selection of at least one of the predefined selectable options which are representative of the raw data. In the case of an optionally selectable field, conversion comprises checking, using the system, whether the optionally selectable field should be selected or unselected, and if it is determined that the field should be selected, then the system selects the same.

Since the format of collected raw data is not typically textual (for example, usually audio or visual), the step of converting the raw data to a suitable form for input to the system-decided data field may comprise an initial step of transcribing the raw data to text, which is stored so as to be available for user-verification of representativeness of the text to the raw data, and a subsequent step of selecting an excerpt of at least one word from the converted text that is determined by the system to be relevant to the type of health information associated with the data field. After the step of selecting a relevant excerpt, there is an optional step of converting the textual excerpt to the format of the data field if the format is not free-form text.

As the raw data is converted to the appropriate form and input to the data fields, or after all raw data has been converted and input to the data fields, the system displays at least one confidence value, determined by the system, which represents a predicted accuracy about how well the converted data represents the raw data from which it was derived. Basically, it is appreciated that the system employs conversion methods in the form of machine learning algorithms that are not determinative, meaning that there exists a possibility of multiple solutions (results of conversion) for the same starting set of raw data. This confidence value is based on the data acquisition device with which the raw data was originally captured, meaning that the confidence value is at least partially based upon the format of the raw data and at least partially on a complexity of conversion of that raw data to the format of the data field associated with the classified type of health information.

For example, the confidence value for audio data relates to transcription thereof to text, which may be on a word-by-word basis.

For example, the confidence value for image data relates to the scanning process and interpretation of the article(s) in the image, such as a label of a personal medication container.

For example, captured location data may also receive a confidence value which relates to a type of location system.

For example, the confidence value for data from a medical diagnostic device relates to the classification process of the data collected from the medical diagnostic device.

The converted data in each data field in the electronic health record is provided a confidence value CF typically displayed adjacent or otherwise in association with the data field containing the converted data. Thus, in FIG. 2, there is provided a distinct one or set of confidence values CF₁ through CFₙ for each data field DF₁ through DFₙ.

Typically, at step 28, the system additionally displays a confidence value that is representative of a predicted accuracy of the type of health information of the raw data, as classified by the system, to the actual type of health information with which the raw data is associated, since this classification determines placement of the converted data in the electronic patient record which is also related to accuracy of populating the health record.

In the event that the system cannot classify the raw data according to a type of health information, or that the determined confidence value (corresponding to classified type of health information) is below a prescribed minimum threshold, then the system may skip the step of assigning the raw data to one of the data fields and defer assignment to the user, for example when the system during a user-verification step of the assigned collected data.

The system is configured to calculate for the user's reference two average levels of confidence, a first associated with the representativeness of the converted data to the raw data, and a second associated with placement of the converted data in an appropriate data field in the electronic health record.

In a configuration of the system where raw data is transcribed to text, the system is configured to determine and display to the user, for each processing step of a piece-meal conversion to the format for input to the data field, a confidence value representative of a predicted accuracy of the converted data relative to the data prior to the processing step. This confidence value is based on the processing technique or algorithm applied in order to render the processed data. Thus, the step of converting raw data may include assigning one or more confidence values to intermediate processing steps of the conversion. The confidence values of the intermediate processing steps may be displayed to the user, as distinct from the confidence value corresponding to the final result of the conversion, as entered into one of the data fields. It will be appreciated that the confidence value of the final result of the conversion process is a combination of, or otherwise incorporates or factors in, the confidence values of each intermediate processing step.

For example, when sentences detected in raw audio data are analyzed, which analysis includes classifying sentence types, then a confidence value therefor relates to the classification process into sentence types.

For example, when sentences detected in raw audio data are analyzed, which analysis includes categorizing sentence each sentence according to a predetermined list of the types of health information about the patient, then a confidence value therefor relates to the classification process into types of health information.

For example, when sentences detected in raw audio data are analyzed, which analysis includes grouping sentences detected as questions and sentences detected as related responses and correlating the sentences in the grouping to determine the context of the situation represented by the raw data, then a confidence value therefor relates to the classification process into groupings.

For example, when sentences detected in raw audio data are analyzed to determine a speaker, which includes analyzing voice patterns in the detected sentences to distinguish a plurality of speakers, then a confidence value therefor relates to the classification process of speaker determination.

In a scenario when one of the data fields DF₁ through DFₙ contains converted data from multiple data acquisition devices, part of displaying a confidence value representing predicted accuracy of the input data comprises, after having determined that the data field contains multiple source data, comparing the converted data from the multiple data acquisition devices to determine whether information represented by said converted data is consistent, and if the corresponding data field is determined to contain information that is inconsistent, flagging for the user's review, using a visual marker, the corresponding data field.

The step of determining and displaying confidence values at 28 further comprises a step, indicated at 38, of flagging for the user's review, using a visual marker, the corresponding data field into which the converted data was input when at least one of the confidence values is below a prescribed threshold value of the system that is associated with that confidence value.

A visual marker may comprise, for example, highlighting of the data field with a designated colour. There may be a legend of a plurality of designated colours each corresponding to a different range of confidence values.

In some embodiments, the confidence value is displayed by the highlighting of the designated colour and a numerical value may not be displayed to the user.

In one embodiment, for all of the data fields of the electronic health record or for a subset of the data fields thereof, the prescribed threshold value may be 100%. Thus, generally speaking, all data fields receiving converted data derived from non-deterministic processes, such as machine learning algorithms, are expected to be flagged for review on the basis that at least some user-verification is recommended by the system to ensure the converted data is representative of the original raw data.

In another embodiment, for all of the data fields of the electronic health record or for a subset of the data fields thereof, the prescribed threshold value is defined by input to the system. The input can be provided by a manufacturer of the system or by the user or by an entity by which the user is employed.

Furthermore, as the system is configured to capture location data, there may be provided a step of displaying to the user a confidence value representative of a predicted accuracy of the captured or measured location data relative to an actual location associated therewith or corresponding thereto. This confidence value is typically based on the data acquisition device with which the location data was collected.

Following display of confidence values and subsequent flagging of data fields in which the system determines it is not certain about representativeness of the converted data relative to the raw data, the next step of user-verification at 31 generally comprises:

providing, using the system, the raw data, as well as the textual transcription thereof (as applicable), to the user for comparison to verify the converted data input to the data fields of the electronic health record, as at step 40; and

requesting input, from the user, to confirm that the converted data input to the data fields of the electronic health record are representative of the raw data captured by the data acquisition devices and actual types of health information associated therewith, as at step 42.

In regard to step 40, verification of the representativeness of the converted data relative to the raw data is determined by the user by comparing the raw data made available for review by the system. For example, when the raw data is audio data, this means providing an audio clip associated with the raw data for playback by the user. If the raw audio data was transcribed as part of the data capture step, and the data field has an input form other than free-from text, the transcribed text is provided additionally or alternatively to the audio clip. In another example, when the raw data is image data, step 40 comprises displaying to the user an image associated with the raw data.

At step 42, requesting input to correct the converted data generally comprises receiving input, from the user, to correct the converted data to be representative of the raw data. Thus the user is enabled by the system to manually amend the input to the data field.

Coinciding with the user-verification step the system may perform a training step 45, on itself, based on corrections to the converted data made by the user. That is, the system updates or revises the non-deterministic machine learning algorithms employed thereby to convert the raw data to a form for input to the corresponding health record data field.

As such, the confidence values which are determined by the system are based on an initial predetermined value associated with the corresponding data acquisition device, used to capture the original raw data, and previous user confirmations of converted data based on raw data captured by the corresponding data acquisition device. For example, each data acquisition device may have an initial starting confidence value which is fixed and predefined by the system, and the confidence value displayed to the user is determined by an equation accounting for this starting value and recent evaluations of the success of the algorithms, as assessed by the user, in conversion and placement of data into the health record.

Once all of the data fields which need input have been verified for the user, the electronic health record is saved on the system either locally on the computing device 2 or at a remote storage device which is part of or associated with the system, as at step 48. The raw data which was saved so as to be made available to the user is typically deleted by the system after user-confirmation at step 50 in accordance with local privacy legislations, generally in conjunction with the aforementioned step of saving the health record.

Thus is provided a system and method for populating an electronic health record in a manner which provides to the user a story of a medical or health-related event that is auditable. This expedites the data acquisition and entry steps which are performed automatically by the system. As it is appreciated that in some instances non-deterministic processes such as machine learning algorithms are used to convert raw data to a form for input to data fields in the electronic health record, the system provides the user with an opportunity to audit or verify the automatically entered data. The system suggests to the user, by display the confidence values, an amount of scrutiny for reviewing the data in a field of the electronic health record. In order to perform this audit the system temporarily stores and makes available for retrieval by the user the raw data that was originally captured. Thus, the raw data converted to input to a respective data field is made available for retrieval by display adjacent or otherwise in association with the corresponding data field and is thus represented by box DT₁ through DTₙ in FIG. 2.

The computing device 2 on which the electronic health record 1 is at least temporarily stored is used to perform the steps of displaying the confidence value to the user and user-verification.

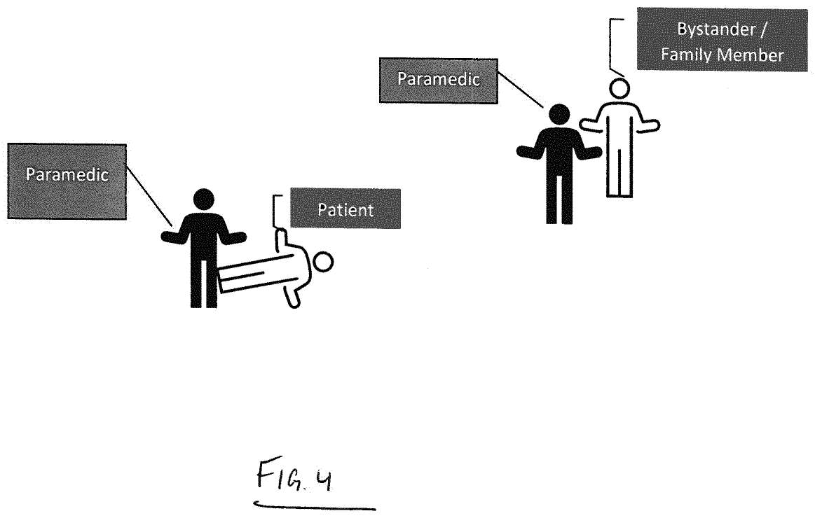

The step of converting the raw data captured by the corresponding data acquisition device to a form for input to a corresponding data field is performed by the corresponding data acquisition device. This provides distributed processing within the system where multiple formats of raw data may be simultaneously acquired (see FIG. 4 showing two paramedics, one attending to a patient and the other interviewing a bystander). Thus each data acquisition device such as 7 and 8 comprises a processor and non-transitory memory operatively coupled thereto storing instructions to (i) store raw data, (ii) classify the raw data according to health information type and (iii) convert the raw data to the input as determined by the data field associated with the classified type of health information.

Each data acquisition device is also configured to determine the confidence values and to provide these to the computing device. Thus the memory thereof stores executable instructions to analyze the captured raw data with a non-deterministic machine learning algorithm.

In other words, in such configurations the memory of each data acquisition device is configured to store executable instructions thereon to:

classify raw data captured by the data acquisition device according to a type of health information by analysis of said raw data;

convert the raw data that has been captured to a form for input to a corresponding one of the data fields of the electronic health record associated with the classified type of health information of said raw data; and

determine a confidence value representative of a predicted accuracy of the converted data to the raw data.

There is also described herein a method for forming a database of electronic health records containing information which is searchable because all of the converted data is in the form of either textual transcription of the raw data, system-selection of a predefined selectable option in a predetermined list, or system-selection of an optionally selectable field. Thus is provided a method for optimizing a global database of electronic health records because the information contained therein is in a standardized format that is searchable.

As described hereinbefore the present invention relates to a method for populating an electronic health record, and a system configured to perform this method, comprising a step of analyzing captured raw data to classify the same according to type of health information to which it pertains; a step of converting the raw data to a format based on input type of a data field which is associated with the classified type of health information; determining and displaying confidence values representative of predicted accuracies of the converted text relative to the raw data and of selection of the data field receiving the converted text; and requesting user-verification that the converted data is representative of the raw data.

The electronic health record may be any medical chart where health information of a patient can be input, for example at triage to an emergency department of a hospital.

The scope of the claims should not be limited by the preferred embodiments set forth in the examples but should be given the broadest interpretation consistent with the specification as a whole.

1. A method for populating an electronic health record using a system comprising a computing device and a plurality of data acquisition devices communicatively coupled to the computing device,
wherein the computing device comprises a processor, a memory operatively coupled thereto and configured to store executable instructions thereon, and a visual display configured for displaying information to a user,
wherein the computing device is configured to receive input from the user,
wherein the electronic health record is stored on the memory of the computing device,
wherein the electronic health record comprises a plurality of data fields configured for receiving input, each data field being associated with a different type of health information about a patient,
the method comprising:
after capturing, using at least one of the data acquisition devices, raw data about the patient, classifying, using the system, for the raw data captured by a corresponding one of the data acquisition devices, said raw data according to a type of health information by analysis of said raw data;
using the system, converting the raw data captured by the corresponding data acquisition device to a form for input to a corresponding one of the data fields associated with the classified type of health information of said raw data;
using the system, displaying to the user a confidence value representative of a predicted accuracy of the converted data input to the corresponding data field relative to the raw data captured by the corresponding data acquisition device, wherein the confidence value is based on the data acquisition device with which the raw data was captured;
using the system, providing the raw data to the user for comparison to verify the converted data input to the data fields of the electronic health record; and
using the system, requesting input, from the user, to confirm that the converted data input to the data fields of the electronic health record are representative of the raw data captured by the data acquisition devices and actual types of health information associated therewith.

2. The method of claim 1 further including displaying, using the system, for the raw data captured by the corresponding data acquisition device, a confidence value to the user that is representative of a predicted accuracy of the classified type of health information of said raw data to the actual type of health information with which said raw data is associated, wherein the confidence value is based on the data acquisition device with which the raw data was captured.

3. The method of claim 1 further including capturing, using the system, the raw data for subsequent conversion and input to the electronic health record, and wherein capturing the raw data comprises recording audio and transcribing the audio to text in real-time.

4. The method of claim 1 further including capturing, using the system, the raw data for subsequent conversion and input to the electronic health record, and wherein capturing the raw data comprises scanning a unique identifier of a personal identification document of the patient.

5. The method of claim 1 further including capturing, using the system, the raw data for subsequent conversion and input to the electronic health record, and wherein capturing the raw data comprises capturing an image of a label on a personal medication container of the patient.

6. The method of claim 1 further including capturing, using the system, the raw data for subsequent conversion and input to the electronic health record, and wherein capturing the raw data comprises capturing an image of medication to be administered to the patient.

7. The method of claim 1 further including capturing, using the system, location data from at least one of the data acquisition.

8. The method of claim 1 wherein displaying to the user a confidence value representative of a predicted accuracy of the converted data comprises flagging for the user's review, using a visual marker, the corresponding data field into which the converted data was input when the confidence value is below a prescribed threshold value of the system.

9. The method of claim 8 wherein the prescribed threshold value is 100%.

10. The method of claim 8 wherein the prescribed threshold value is defined by input to the system.

11. The method of claim 1 further including:
using the system, checking if one of the data fields of the electronic health records contains converted data from multiple ones of the data acquisition devices, and
if one of the data fields contains converted data from multiple ones of the data acquisition devices:
comparing the converted data from the multiple data acquisition devices to determine whether information represented by said converted data is consistent, and
if the corresponding data field is determined to contain information that is inconsistent, flagging for the user's review, using a visual marker, the corresponding data field.

12. The method of claim 1 wherein, when the raw data captured by the corresponding data acquisition device is audio data, at least one of steps of (i) classifying the raw data according to a type of health information associated therewith and (ii) converting the raw data to a form for input to a corresponding one of the data fields associated with the classified type of health information comprises analyzing, using the system, sentences detected in the raw data to determine at least one of a context and a speaker of a situation represented by the raw data.

13. The method of claim 12 wherein analyzing sentences detected in the raw data comprises classifying sentence types of the detected sentences.

14. The method of claim 13 wherein analyzing sentences detected in the raw data comprises categorizing each sentence according to a predetermined list of the types of health information about the patient.

15. The method of claim 13 wherein analyzing sentences detected in the raw data further comprises grouping sentences detected as questions and sentences detected as related responses and correlating the sentences in the grouping to determine the context of the situation represented by the raw data.

16. The method of claim 12 wherein analyzing sentences detected in the raw data to determine a speaker comprises analyzing voice patterns in the detected sentences to distinguish a plurality of speakers.

17. The method of claim 1 further including, when the raw data captured by the corresponding data acquisition device is audio data and when the form of the converted data is text, checking, using the system, at least one of spelling and grammar of said converted data for classifying context of the situation represented by said raw data.

18. The method of claim 1 wherein, when the raw data captured by the corresponding data acquisition device is audio data, providing the raw data to the user for comparison to verify the converted text comprises providing an audio clip associated with said raw data for playback by the user.

19. The method of claim 1 wherein, when the raw data captured by the corresponding data acquisition device is image data, providing the raw data to the user for comparison to verify the converted text comprises displaying to the user an image associated with said raw data.

20. The method of claim 1 further including automatically attaching to the electronic health record data collected from a medical diagnostic device that is operatively communicated with the system.

21. The method of claim 1 further including, using the system, deleting the raw data after user-confirmation of the data fields of the electronic health record.

22. The method of claim 1 wherein requesting input, from the user, to confirm that the converted data input to the data fields of the electronic health record are representative of the raw data captured by the data acquisition devices and actual types of health information associated therewith comprises receiving input, from the user, to correct the converted data to be representative of the raw data.

23. The method of claim 22 further including training the system based on corrections to the converted data made by the user.

24. The method of claim 1 wherein the confidence value representative of a predicted accuracy of the converted data input to the corresponding data field relative to the raw data captured by the corresponding data acquisition device is based on an initial predetermined value associated with the corresponding data acquisition device and previous user confirmations of converted data based on raw data captured by the corresponding data acquisition device.

25. The method of claim 1 wherein converting the raw data captured by the corresponding data acquisition device to a form for input to a corresponding one of the data fields is performed by the corresponding data acquisition device.

26. The method of claim 1 wherein the confidence value representative of a predicted accuracy of the converted data input to the corresponding data field relative to the raw data captured by the corresponding data acquisition device is provided to the computing device by the corresponding data acquisition device.

27. The method of claim 1 wherein converting the raw data captured by the corresponding data acquisition device comprises transcribing the raw data to text.

28. The method of claim 27 wherein converting the raw data captured by the corresponding data acquisition device further comprises selecting an excerpt of the transcribed text associated with the classified type of health information of the raw data.

29. The method of claim 28 wherein converting the raw data captured by the corresponding data acquisition device further comprises assigning a confidence level to the classified type of health information.

30. The method of claim 27 wherein converting the raw data captured by the corresponding data acquisition device further comprises converting the textual excerpt to the form for input to the corresponding data field.

31. A system for populating an electronic health record, wherein the electronic health record comprises a plurality of data fields for receiving input, each field being associated with a different type of health information about a patient,
the system comprising:
a portable computing device having a processor, a memory operatively coupled thereto and configured to store executable instructions thereon, and a visual display configured for displaying information to a user;
wherein the portable computing device is configured to receive input from the user,
wherein the memory of the portable computing device is configured to store the electronic health record thereon;
a plurality of data acquisition devices communicatively coupled to the portable computing device;
wherein at least one of the data acquisition devices is arranged to be located on a body of the user so as to be worn;
wherein each data acquisition device has a processor and a memory operatively coupled thereto and configured to store executable instructions thereon to:
classify raw data captured by the data acquisition device according to a type of health information by analysis of said raw data;
convert the raw data that has been captured to a form for input to a corresponding one of the data fields of the electronic health record associated with the classified type of health information of said raw data; and
determine a confidence value representative of a predicted accuracy of the converted data to the raw data.
